(19) United States
(12) Patent Application Publication
(10) Pub. No.: US 2014/0356433 A1
(43) Pub. Date: Dec. 4, 2014

(54) Pharmaceutical Carrier and Drug Structure Using the Same

(71) Applicant: MING-JIUM SHIEH, TAIPEI CITY (TW)

(72) Inventors: Ming-Jium SHIEH, Taipei (TW); Yuan-Ting CHANG, Taipei (TW)

(73) Assignee: MING-JIUM SHIEH, TAIPEI CITY (TW)

(21) Appl. No.: 14/087,088

(22) Filed: Nov. 22, 2013

(30) Foreign Application Priority Data
Jun. 4, 2013 (TW) 102119750

(51) Int. Cl. A61K 47/36; A61K 31/43

(52) U.S. Cl. CPC A61K 47/36; A61K 31/43
USPC 424/489; 514/197; 514/777

(57) ABSTRACT

The present invention provides a pharmaceutical carrier and a drug structure using the carrier. The drug of the present invention comprises particular contents of chitosan, a negatively charged polymer, sodium tripolyphosphate, and an active ingredient, which combine with each other via electrostatic attraction. The drug structure has better release property and longer retention time; therefore overcomes the current drawbacks of the conventional treatment.

### RELATED APPLICATIONS

The application claims priority to Taiwan Application Serial Number 102119750, filed Jun. 4, 2013, which is herein incorporated by reference.

### BACKGROUND

1. Technical Field

The present invention relates to a drug structure and applications thereof; more particularly, a drug structure for inhibiting Helicobacter pylori.

2. Description of Related Art

Helicobacter pylori (in short, H. pylori) are a kind of bacteria living inside stomachs and duodenums. At the beginning, most of the scientists did not believe that any microorganism can stand and live in the extremely acid environment of gastric fluid. Along with the accumulation of researches, the National Institutes of Health (NIH) of United States formally reported in 1994 that most of the common stomach diseases or inflammation conditions results from H. pylori, and recommended the usage of antibiotics in treatments. So far, it is well known that H. pylori are highly related to chronic gastritis, gastric ulcer, duodenal ulcer, etc. In addition, the World Health Organization has announced H. pylori as one of the microorganism-based cancerogenic substance, which is the first cancerogenic prokaryote identified.

Gastric ulcer resulting from H. pylori is very common for people of present time. H. pylori are recognized as quite tough bacteria; therefore it is never easy to treat gastric ulcer. In order to root H. pylori out, triple therapy with antibiotics and proton pump inhibitor is the most clinical-proved effective treatment by now and the best treatment strategy for avoiding recurrence. However, using various kinds of medicines for one treatment may have the risk of causing several undesired side effects, and the compliance of patients in taking medicines is another big challenge in the treatment.

Taiwanese Patent Publication No. 201138788 discloses a shell-core drug structure comprising alginate as a substrate for encapsulating active ingredient to form a microsphere and a chitosan coating outside said microsphere for forming the disclosed drug structure. According to its specification, the drug structure takes the advantage of the inherent properties of alginate to obtain a slow-releasing effect. The disclosed drug structure may be favorable for protecting drug from being destroyed by gastric fluid but is not helpful for resolving the drawbacks caused by having to use multiple drugs in the gastric ulcer treatment.

U.S. Pat. No. 6,284,745 discloses a drug structure having alginate and chitosan. The preparation of the drug structure also contains the addition of calcium pantothenate for enhancing alginate forming colloid beads for encapsulating drugs. The drug structure has a rapid-releasing feature that can release the contained active ingredient within 2 hours, but it is also not helpful for resolving the drawbacks caused by multiple drugs usage in the gastric ulcer treatment.

To sum up, improvements in drug structure may be one of the ways out for breaking through the current difficulties in gastric ulcer treatment. Both of the aforesaid Taiwanese Patent Publication and US patent teaches drug structures adapting the usage of alginate and chitosan but having different properties from each other and still have some room for improvements. It is noted that although alginate and chitosan are both potential materials for drug carrier preparation, parameters such as the percentages of ingredients, structure and method for combining ingredients, size of the carrier obtained, significantly influence the efficacy and properties of the drug structure concerned. More researches and breakthrough are required in this regard.

### SUMMARY

In light of the foregoing, one of the objects of the present invention is to provide a pharmaceutical carrier, whose ingredients are biocompatible and favorable for drug release or extending the retention time of active ingredient in the body, and thus improving drug efficacy.

Another object of the present invention is to provide a drug structure and a method for inhibiting Helicobacter pylori by using the same; wherein, by virtue of the ingredient selection for the drug structure, the effects of the drug can be performed even more completely and better efficacy can be achieved.

Another object of the present invention is to provide a preparation method of a drug structure, which is not only simple but also capable of obtaining improved efficacy to the drug prepared.

In order to achieve the aforesaid objects, the present invention provide a pharmaceutical carrier, prepared by an initial mixture, wherein said initial mixture comprises: 100 parts of a negatively charged polymer; 625 to 5000 parts of chitosan; and 250 to 2000 parts of sodium tripolyphosphate; wherein, in said pharmaceutical carrier, said negatively charged polymer, said chitosan, and said tripolyphosphate combine with each other via electrostatic attraction.

Preferably, said initial mixture comprises: 100 parts of a negatively charged polymer; 2400 to 2600 parts of chitosan; and 900 to 1100 parts of sodium tripolyphosphate.

Preferably, said pharmaceutical carrier has a particle size of 100 to 200 nm.

Preferably, said pharmaceutical carrier is used for carrying drug for inhibiting Helicobacter pylori.

Preferably, said negatively charged polymer is alginate, heparin, polyacrylic acid, or a combination thereof.

The present invention also provides a drug structure, prepared by an initial mixture, wherein said initial mixture comprises: 100 parts of a negatively charged polymer; 625 to 5000 parts of chitosan; 250 to 2000 parts of sodium tripolyphosphate; and 500 to 4000 parts of an active ingredient wherein, in said pharmaceutical carrier, said negatively charged polymer, said chitosan, said tripolyphosphate, and said active ingredient combine with each other via electrostatic attraction.

Preferably, said initial mixture comprises: 100 parts of a negatively charged polymer; 2400 to 2600 parts of chitosan; 900 to 1100 parts of sodium tripolyphosphate; and 1800 to 2200 parts of an active ingredient.

Preferably, said drug structure has an encapsulation efficiency of 45 to 55%.

Preferably, said drug structure has a particle size of 100 to 200 nm.

Preferably, said negatively charged polymer is alginate, heparin, polyacrylic acid, or a combination thereof.

Preferably, said drug structure does not comprise a proton pump inhibitor or a bismuth.

Preferably, said active ingredient has an activity of inhibiting Helicobacter pylori.

Preferably, said active ingredient is selected from amoxicillin, clarithromycin, or omeprazole.

Preferably, said drug structure has a retention time in stomach of no less than 24 hours.

Preferably, said drug structure has a surface charge of 20 to 30 mV.

Preferably, said drug structure is an oral medicine.

The present invention also provides a method for inhibiting Helicobacter pylori, comprising administrating an effective amount of said drug structure to a population of Helicobacter pylori.

Preferably, said method consists essentially of the following step: administrating an effective amount of said drug structure to a population of Helicobacter pylori.

Preferably, said method does not comprise administrating another substance having an activity of inhibiting Helicobacter pylori to said population of Helicobacter pylori; wherein said substance having an activity of inhibiting Helicobacter pylori is different from said active ingredient of said drug structure.

Preferably, said method does not comprise administrating a substance having an auxiliary activity of inhibiting Helicobacter pylori to said population of Helicobacter pylori; wherein said substance having an auxiliary activity of inhibiting Helicobacter pylori is different from the ingredients of said drug structure.

Preferably, said substance having an auxiliary activity of inhibiting Helicobacter pylori is proton pump inhibitor or bismuth.

Preferably, said effective amount is 1 to 10 mg/kg/day.

To sum up, the pharmaceutical carrier and drug structure of the present invention contain particular components in particular amount that are favorable for releasing active ingredient and extending the retention time thereof in an organism. Therefore, the efficacy of the drug can be performed more completely. Moreover, the drug structure of the present invention is designed to take the virtue of the electric properties of each component for combining with each other via electrostatic force. In other words, the drug structure of the present invention is a structure formed by mixing of the components thereof but not a core-shell structure; therefore, the preparation thereof is way easier than that of the conventional drugs in the form of core-shell structures.

### BRIEF DESCRIPTION OF THE DRAWINGS

FIG. 1 displays the TEM images of the present drug structure in various pH environments; (A) pH=1.2; (B) pH=4.5; (C) pH=7.4.

FIG. 2 shows the releasing curve of the active ingredient of the present drug structure in various pH environments.

FIG. 3 shows the in vivo position of the ¹²³I labeled amoxicillin and the present drug structure inside a mouse; (A) amoxicillin, 1 hour after feeding, whole body image was taken; (B) amoxicillin, 4 hours after feeding, whole body image was taken; (C) amoxicillin, 24 hours after feeding, whole body image was taken; (D) amoxicillin, 24 hours after feeding, gastrointestinal tract image was taken; (E) the present drug structure, 1 hour after feeding, whole body image was taken; (F) the present drug structure, 4 hours after feeding, whole body image was taken; (G) the present drug structure, 24 hours after feeding, whole body image was taken; (H) the present drug structure, 24 hours after feeding, gastrointestinal tract image was taken.

FIG. 4 shows the activities of amoxicillin, the present pharmaceutical carrier and the present drug structure in inhibiting Helicobacter pylori.

### DETAILED DESCRIPTION

The drug structure of the present invention has particular components in particular percentages; therefore is favorable for improving the drug efficacy. Moreover, by using the drug structure of the present invention, an excellent effect in inhibiting Helicobacter pylori can be obtained while one single drug is used. It is a breaking through for the conventional treatments of gastric ulcer, which uses several active ingredients (drug) along with a proton pump inhibitor.

The activity of “inhibiting Helicobacter pylori” as mentioned in the present invention means, in macroscopic view, an activity of “controlling the size of a Helicobacter pylori population, reducing a Helicobacter pylori population, and/or disappearing Helicobacter pylori population”; in microscopic view, an activity of “reducing the physiological status of Helicobacter pylori, reducing the virulence of Helicobacter pylori, and/or killing Helicobacter pylori”.

The “substance(s) having activity of inhibiting H. pylori” mentioned in the present invention is referred to a substance having the activity of “inhibiting Helicobacter pylori” as recited in the preceding paragraphs. More specifically, an example of the substance(s) is antibiotics, which include but not limited to Amoxicillin, Clarithromycin or Omeprazole. More specifically, the “active ingredient” recited in the present invention is a substance having activity of inhibiting H. pylori.

The “substance(s) having auxiliary activity of inhibiting H. pylori” mentioned in the present invention is referred to a substance which has no direct activity of “inhibiting Helicobacter pylori” as recited in the preceding paragraph but is favorable for the “substance(s) having activity of inhibiting H. pylori” to conduct its efficacy. More specifically, in the conventional treatment for gastric ulcer, not only triple therapy of antibiotics but also proton pump inhibitor is required. The proton pump inhibitor has no direct activity of inhibiting Helicobacter pylori but auxiliary improves the efficacy of the antibiotics. In other words, the proton pump inhibitor is a kind of substances having auxiliary activity of inhibiting H. pylori.

However, the ordinary artisan shall appreciate that the “substance(s) having auxiliary activity of inhibiting H. pylori” mentioned in the present invention does not include substances designed based on Pharmacology for assisting the applying of drugs, improving the flavor of drugs, or prolonging the storage time of drugs. For instance, the “substance(s) having auxiliary activity of inhibiting H. pylori” mentioned in the present invention does not include additives that are commonly used in a drug structure such as a pharmaceutical carrier, a flavor, or a preservative, etc.

Except for the aforesaid proton pump inhibitor, another example of “substance(s) having auxiliary activity of inhibiting H. pylori” includes a bismuth.

A method for preparing the drug structure of the present invention comprises the following steps. Firstly, preparing every component required for the drug structure of the present invention, including a chitosan, a negatively charged polymer, a sodium tripolyphosphate and an active ingredient. After all the required components are prepared, mixing said chitosan, said negatively charged polymer, said sodium tripolyphosphate and said active ingredient to form a initial mixture, then letting all the components of the initial mixture react with each other at 25° C. for 15 to 30 minutes to obtain the drug structure of the present invention.

Chitosan is a natural polymer of 2-amino-2-deoxy-β-D-glucan via glycosidic linkages, which can be obtained by transferring the acetyl groups of chitin into amino groups via deacetylation. Chitosan is positive charged in acidic environment and is able to combine with other charged components (such as drugs) via electrostatic attraction. Chitosan is widely used as a material for pharmaceutical carriers because of its superior biocompability, mucoadhesive and bacteriostatic. Preferably, the chitosan used in the present invention has a molecular weight of 3,800 to 150,000 Da and a deacetylation degree of 66 to 99%.

Said negatively charged polymer is referred to a polymer having negative charge in neutral or acidic environment; for instance, a polymer having negative charge in an environment of pH value of 1 to 8; preferably, a polymer having negative charge in environment of pH value of 2 to 7. Said negatively charged polymer includes but not limited to alginate, heparin or polyacrylic acid. Preferably, said negatively charged polymer is alginate.

Said active ingredient is referred to any compound that is to achieve the objects of treatment, prevention, or inspection. In some of the embodiment of the present invention, said active ingredient is particularly referred to compound that used for gastric ulcer treatment; that is, the aforesaid substance(s) having activity of inhibiting H. pylori, including amoxicillin, clarithromycin, or omeprazole. Preferably, the present drug structure only uses a single kind of said substance having activity of inhibiting H. pylori as active ingredient. Preferably, the present drug structure does not have said substance having auxiliary activity of inhibiting H. pylori.

Preferably, said chitosan, said negatively charged polymer, said sodium tripolyphosphate, and/or said active ingredient is in a form of solution, which is favorable for controlling the pH value of each component so that maintaining each component at proper charged state.

Preferably, said chitosan is in a form of solution, which has a pH value of 3 to 4; said negatively charged polymer is alginate, heparin, or polyacrylic acid; said negatively charged polymer is in a form of solution, which has a pH value of 6 to 7; said sodium tripolyphosphate is in a form of solution, which has a pH value of 6 to 7; said active ingredient is in a form of solution, which has a pH value of 4 to 5.

The order for performing said mixing is not limited, but preferably, said negatively charged polymer is mixed with said sodium tripolyphosphate then said chitosan and said active ingredient are added. Alternatively, said negatively charged polymer, said sodium tripolyphosphate and said chitosan are mixed and fabricated as the pharmaceutical carrier of the present invention by the following steps, and then, depending on the need, the pharmaceutical carrier as fabricated before is combined and reacted with said active ingredient to obtain the drug structure.

In one embodiment for preparing the pharmaceutical carrier (that is, not comprising active ingredient) of the present invention, said initial mixture comprises 100 parts of a negatively charged polymer, 625 to 5000 parts of chitosan, and 250 to 2000 parts of sodium tripolyphosphate. Preferably, said initial mixture comprises 100 parts of a negatively charged polymer, 2400 to 2600 parts of chitosan, and 900 to 1100 parts of sodium tripolyphosphate.

In one embodiment for preparing the drug structure of the present invention, said initial mixture comprises 100 parts of a negatively charged polymer, 625 to 5000 parts of chitosan, 250 to 2000 parts of sodium tripolyphosphate, and 500 to 4000 parts of an active ingredient. Preferably, said initial mixture comprises 100 parts of a negatively charged polymer, 2400 to 2600 parts of chitosan, 900 to 1100 parts of sodium tripolyphosphate, and 1800 to 2200 parts of an active ingredient.

Preferably, in order to maintain each component of the present drug structure at proper charged state for maintaining the structure of the present drug structure, said mixture is in a form of solution with pH value of 4 to 5.

Said chitosan, said negatively charged polymer, said sodium tripolyphosphate, and said active ingredient combine with each other via electrostatic attraction. Preferably, the present pharmaceutical carrier and/or the present drug structure have a particle size of 100 to 200 nm; more preferably, the particle size is 100 to 150 nm. In other words, the present pharmaceutical carrier and/or the present drug structure are nanoparticles so that are favorable for being uptaken in organisms.

Preferable, said drug structure has a surface charge of 20 to 30 mV so that is favorable for extending the retention time in stomach. Preferable, the present drug structure has a retention time in stomach of at least 24 hours; therefore the contacting time between said active ingredient and H. pylori can be extended.

The drug structure prepared according to the present invention's teaching in components and amounts thereof is capable of performing the drug efficacy in a more completely way. Therefore, the present drug structure is able to effectively treat gastric ulcer while only one active ingredient is applied.

The method for inhibiting Helicobacter pylori of the present invention comprises applying an effective amount of said drug structure to an object. Preferably, the method of the present invention consists essentially of applying an effective amount of said drug structure to an object.

Said “consists essentially of” means except for applying an effective amount of said drug structure to the object to be treated, the object to be treated does not need to take any other substances having activity of inhibiting H. pylori or any other substances having auxiliary activity of inhibiting H. pylori. Furthermore, the object to be treated does not need any other treatments that are conducted for inhibiting H. pylori.

However, the present method may comprise other steps that are not for the purpose of inhibiting H. pylori, such as relieving the side effects resulted from drug or assisting the object to be treated to rest.

In a prefer embodiment, while using the drug structure of the present invention for inhibiting H. pylori, said active ingredient of said drug structure consists of one single active ingredient and said drug structure does not comprise another substance having activity of inhibiting H. pylori or substance having auxiliary activity of inhibiting H. pylori.

In a prefer embodiment, while using the drug structure of the present invention for inhibiting H. pylori, there is no need to co-treat the object to be treated with another substance having activity of inhibiting H. pylori or substance having auxiliary activity of inhibiting H. pylori.

In a prefer embodiment, while using the drug structure of the present invention for inhibiting H. pylori, the effective amount of the present drug structure is 1 to 10 mg/kg/day.

The following embodiments are recited for further explaining the advantages of the present invention but not for limiting the claim scope of the present invention.

### Embodiment 1

### Preparation of the Present Pharmaceutical Carrier and Drug Structure

The embodiment was to prepare the present pharmaceutical carrier and drug structure by the aforesaid method and to examine the physical properties such as particle size and surface charge of the drug structure.

Briefly, a chitosan solution with a concentration of 0.5 mg/ml was obtained (dissolved in a 0.01 M of acetic acid; pH=4.0), a alginate solution with a concentration of 0.05 mg/ml was obtained (dissolved in a 0.01 N of NaOH; pH=7.0), a sodium tripolyphosphate solution with a concentration of 0.5 mg/ml was obtained (dissolved in d.d.-water; pH=7.0), and a amoxicillin solution with a concentration of 2 mg/ml was obtained (dissolved in dd-water; pH=7.0). Then, the samples of the embodiment were prepared according to the percentage of amount listed in the following table.

The following table 1 lists the weight percentage of the components of the initial mixture for preparing the pharmaceutical carriers of this embodiment. The following table 2 lists the average particle size, PDI and surface charge of each sample of this embodiment.

TABLE 1
Weight percentage of the components of the initial
mixtures of samples 1 to 5

| | weight percentage of chitosan:alginate:sodium |
|---|---|
| Sample | tripolyphosphate |
| 1 | 50:1:20 |
| 2 | 25:1:10 |
| 3 | 16.7:1:6.7 |
| 4 | 12.5:1:5 |
| 5 | 6.25:1:2.5 |

TABLE 2
Properties of samples 1 to 5

| | Average particle | | surface charge (Zeta |
|---|---|---|---|
| Sample | size (nm) | PDI | potential; mV) |
| 1 | 115.43 ± 1.25 | 0.409 | 29.6 ± 0.86 |
| 2 | 124.47 ± 4.60 | 0.417 | 27.4 ± 0.94 |
| 3 | 139.93 ± 1.41 | 0.412 | 27.3 ± 1.30 |
| 4 | 152.47 | 0.373 | 26.1 |
| 5 | 186.43 | 0.335 | 23.3 |

The following table 3 lists the weight percentage of components of samples A to E of drug structures prepared from sample 1 to 6 of pharmaceutical carriers of this embodiment. The following table 4 lists various properties of sample A to E of this embodiment.

TABLE 3
Weight percentage of components of the initial mixtures of samples A to E

| | weight percentage of chitosan:alginate:sodium |
|---|---|
| Sample | tripolyphosphate:amoxycillin |
| A | 50:1:20:40 |
| B | 25:1:10:20 |
| C | 16.7:1:6.7:13.4 |
| D | 12.5:1:5:10 |
| E | 6.25:1:2.5:5 |

TABLE 4
Properties of samples A to E

| | Average | | surface charge | |
|---|---|---|---|---|
| | particle size | | (Zeta potential; | encapsulation |
| Sample | (nm) | PDI | mV) | efficiency (%) |
| A | 125.8 ± 0.73 | 0.263 | 26.6 ± 0.69 | 52 ± 1.0 |
| B | 143.2 ± 2.37 | 0.297 | 22.8 ± 1.28 | 54.8 ± 1.33 |
| C | 158.9 ± 1.14 | 0.313 | 22.1 ± 0.84 | 47.5 ± 2.34 |
| D | 160.8 ± 0.84 | 0.389 | 21.3 ± 1.94 | 46.2 ± 3.2 |
| E | 178.1 ± 0.69 | 0.391 | 20.7 ± 2.4 | 45 ± 1.2 |

According to the data listed in the set forth table 2 and table 3, both of the present pharmaceutical carrier and drug structure are nanoparticles so that it is expected to have superior uptaking efficiency in organisms. Moreover, as the present pharmaceutical carrier and drug structure are not core-shell particles, they can be prepared by solution-based method; that is, by mixing every component in form of solution and obtaining the present pharmaceutical carrier and drug structure via electrostatic attraction resulted from the charged state of each component. According to the PDI data, the solution-based method not only has the advantage of easy to operate but also can obtain the pharmaceutical carrier and drug structure with narrow particle size distribution; which means, the present pharmaceutical carrier and drug structure have superior homogeneity.

### Embodiment 2

### Adhesive and Releasing Properties of the Present Drug Structure

According the previous researches, the inner environment of a stomach can be roughly distinguished into a gastric acid area (pH=1.2 to 2.5), a mucosal layer of stomach wall (pH=4.5 to 7.0), and a cell layer of stomach wall (pH≈7) by different pH values. The traditional drug design focuses on extending the retention time of drug in stomach. As the pathogens usually accumulate at the area that has more host cells (cell layer of stomach wall), simply extending the retention time of the active ingredient in stomach does not always improve the drug efficacy.

In this embodiment, the appearance features of the present drug structure in environments of various pH values were observed by transmission electron microscope (TEM). Taking sample B prepared in the aforesaid embodiment 1 for examination, the present drug structure was respectively placed in the environments of pH 1.2, pH 4.5 and pH 7.4 for mimicking the gastric acid area, the mucosal layer of stomach wall, and the cell layer of stomach wall, respectively and observed by TEM.

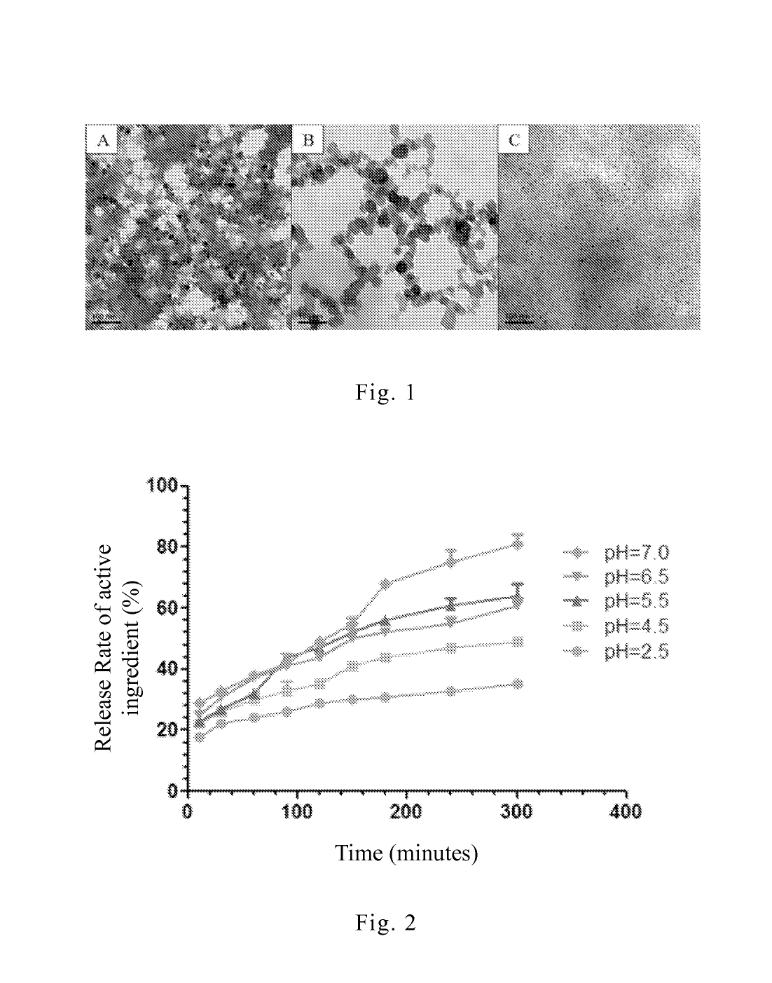

The results of the experiments are shown in FIG. 1. In the gastric acid area (pH=1.2; FIG. 1A), the drug structure was attacked and destroyed by the gastric acid. As both of the chitosan and alginate contained in the present drug structure have the features of adhering to mucosal tissues, the destroyed drug structure tends to adhere to the mucosal layer of stomach wall. The mucosal layer of stomach wall has a pH value of about 4.5 and FIG. 1B of this experiment shows the appearance features of the present drug structure under pH 4.5. It was noted from the FIG. 1B that the present drug structure was most stable under this environment. On the other hand, it was noted that when the present drug structure was at the cell layer of stomach wall (pH=7.4; FIG. 1C), as the charged state of chitosan is changed into neutral state while the pH is raising to neutral, the structure of the present drug structure was turned into loose.

The aforesaid results show that when the present drug structure adhered to the mucosal tissues and is close to the neutral environment of the cell layer of stomach wall, the structure of the drug structure was getting loose because the charged state of chitosan was changed accordingly. Consequently, the active ingredient of the drug structure was released. The releasing pattern enables the active ingredient can be released much more closely to the site where most of the pathogens accumulate so that the efficacy of the active ingredient can be improved.

In order to further understand the releasing pattern of the present drug structure, the sample B of embodiment 1 was concentrated and put into environments of various pH (2.5, 4.5, 5.5, 6.5, 7.0). Then the sample was spun at low speed (150 rpm) and tests were conducted at different time points to detect the concentration of the released active ingredient (HPLC; detecting the concentration of amoxicillin at wavelength 229 nm). The detected concentration was then calculated into release rate.

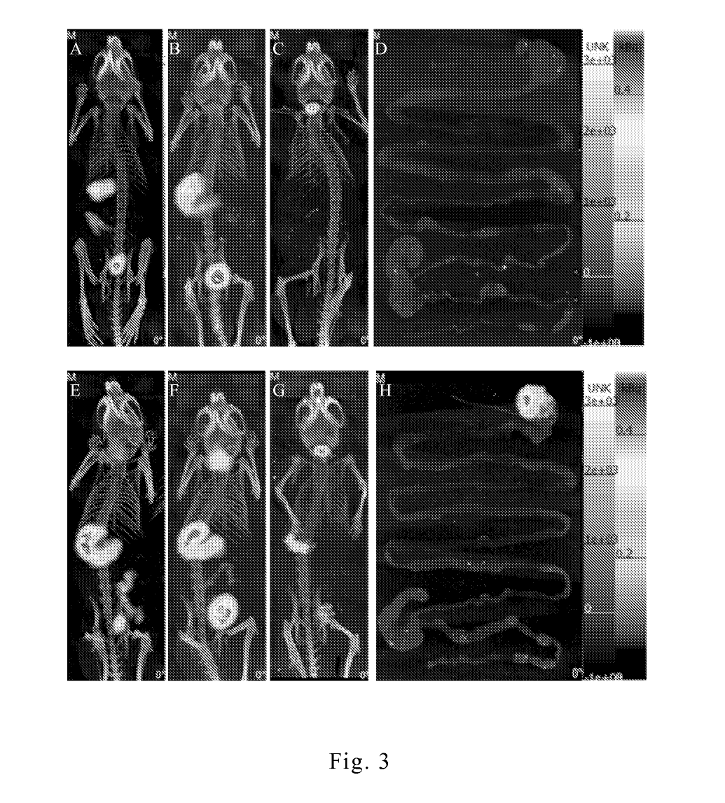

The results of the experiment are shown in FIG. 2. At the same time point, the closer the present drug structure is to neutral (or even basic) environment, the higher the release rate is. Besides, in the period of the experiment, the detection of pH=7 has the highest release rate (over 80%). The results are consistent with the results shown in FIG. 1C; wherein the drug structure became loose as the charged state of chitosan was changed. To sum up, those results support that the present drug structure has superior release rate.

Then, in order to examine the retention time of the present drug structure in stomach, sample B of embodiment 1 was labeled with radioactive Iodine 123 (¹²³I) and fed to experimental mice. Single-photon emission computed tomography was respectively conducted at 1^{st}, 4^{th}, and 24^{th} hour after feeding to observe the location of the present drug structure inside the mice's bodies.

The results of the experiment are shown in FIG. 3; wherein FIG. 3A˜3D show the control group that are performed with 123I labeled amoxicillin, and FIG. 3E˜3H show the experimental group performed with sample B. It was noted from FIGS. 3A, 3B, 3E and 3F that after feeding for one hour, the drug structure had already arrived to stomach and stayed for at least about 4 hours. By comparing FIGS. 3C and 3G, the drug of control group had no longer existed in the stomach after 24 hours from feeding; whereas, the present drug structure still remained in stomach after 24 hours. Furthermore from the images of gastrointestinal tract of FIGS. 3D and 3H, the present drug structure remained at stomach after 24 hours from feeding; whereas, the drug of control group was already gone.

### Embodiment 3

### Test for the Efficacy of the Present Drug Structure

The experimental results of embodiment 2 have shown that the present drug structure can not only remain in stomach for long period (over 24 hours) but also release the active ingredient more closely to the site where the pathogens accumulate. Those properties are theoretically favorable for improving the efficacy of the active ingredient. Therefore, an in vitro experiment and an animal model were conducted respectively in this embodiment for testing the activity of the present drug structure in inhibiting H. pylori.

For the in vitro experiment, suspension liquids of ten different strains of H. pylori were obtained (OD₄₅₀=1). Then, amoxicillin, sample 2 of embodiment 1, and sample B of embodiment 1 were added respectively (50λ). The suspension liquids of H. pylori were cultured for further 3 hours (in an incubator of 37° C. and anaerobic condition), and detection of OD₄₅₀ was conducted for determining the efficacy in inhibiting H. pylori.

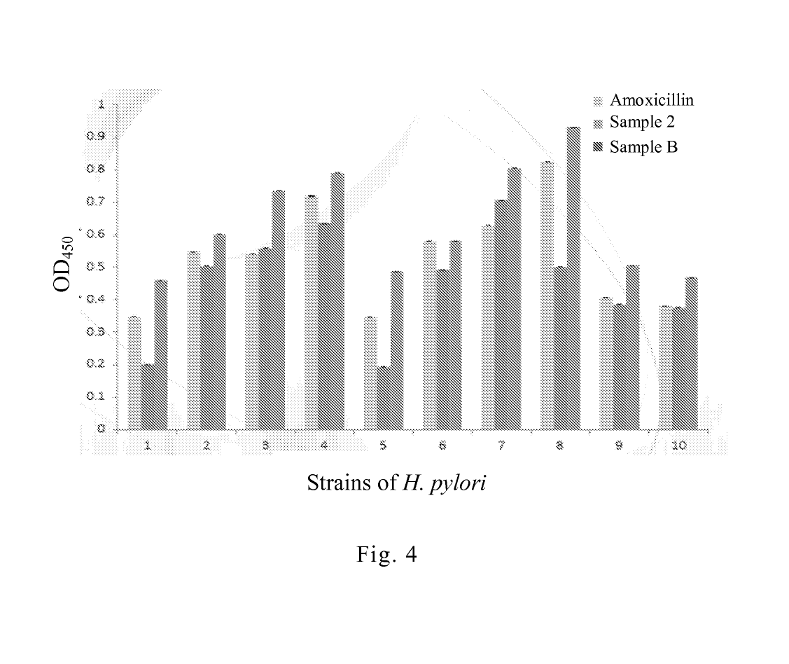

The results are shown in FIG. 4. As different strains of H. pylori have different sensitivity to amoxicillin, different inhibition effects were observed from the groups of amoxicillin. The sample B of the present invention also contains amoxicillin as active ingredient so that a inhibition trend similar with the groups of amoxicillin can be observed; that is, the strain, which was more sensitive to amoxicillin, was also more sensitive to the sample B of the present invention.

In addition, comparing to adding amoxicillin, adding sample B of the present invention had better inhibition effect in 8 out of 10 strains. On the other hand, it was noted from the results that even if containing no active ingredient, the pharmaceutical carrier of the present invention (sample 2) itself had shown slight inhibition activity to H. pylori.

As the advantages of the present drug structure, such as releasing active ingredient closely to the site where the pathogens accumulate and extending the retention time, shall be shown in in vivo experiments, it was expected that the efficacy of the present drug structure can be significantly supported by animal models or clinic trials.

The animal model of this embodiment was conducted with Balb/c mice infected with H. pylori. The animals were fed in the laboratory for 14 days before experiments for adapting the environments. Then, the animals were separated into nine groups and respectively fed with a certain amount of amoxicillin, sample B of the present invention, and sample 2 of the present invention (with or without a proton pump inhibitor; (PPI)). After conducting the experiment for 14 days, the animals were sacrificed and the tissues of the stomachs thereof were gathered and cultured. Then, a polymerase chain reaction was conducted to detect if there was any H. pylori existing in the stomach tissue culture (by detecting the existence of CagA gene). Health mice were used as negative control and mice infected with H. pylori but having not yet been treated were used as positive control in this experiment.

The conditions and results of the experiments of each group are listed in the following table 5. The dosage indicated in the table represents the dosage of the drug used, and 1× (1 fold) dosage was 10 mg/kg/Day, which was referred to the dosage of amoxicillin currently applied for human. The Value of n represents the numbers of experimental mice used. The minus sign (−) means no H. pylori was detected. The positive sign means a H. pylori was detected, and the number indicated in the brackets represent the numbers of the mice being detected to have H. pylori.

TABLE 5
Results of the animal model

| Group | Drug | Dosage | Detection |
|---|---|---|---|
| Negative control (n = 5) | no | no | − |
| Positive control (n = 5) | no | no | + |
| 1 (n = 10) | Amoxicillin + PPI | 1x | − |
| 2 (n = 10) | Sample B | 1x | − |
| 3 (n = 10) | Sample B + PPI | 1x | − |
| 4 (n = 10) | Sample 2 | 1x | |
| 5 (n = 10) | Sample 2 + PPI | 1x | |
| 6 (n = 10) | Amoxicillin + PPI | ½x | − |
| 7 (n = 10) | Sample B | ½x | − |
| 8 (n = 10) | Amoxicillin + PPI | 1/10x | + (3) |
| 9 (n = 10) | Sample B | 1/10x | + (1) |

According to the results listed in the set forth table 5, sample B of present invention alone was sufficient to effectively treat the infection of H. pylori. Furthermore, by comparing the results of Group 8 and Group 9, it is noted that sample B was more effective than the conventional treatment (amoxicillin+PPI) even at 1/10× dosage. These results show the various advantages of the present drug structure (including releasing the active ingredient at site where the pathogens accumulate, and extending the retention time of the active ingredient). The advantages of the present drug structure enable to less usage of drugs than conventional treatment; therefore can save the treatment cost and reduce the risk of side effects.

What is claimed is:

1. A pharmaceutical carrier, prepared by an initial mixture, wherein said initial mixture comprises:
100 parts of a negatively charged polymer;
625 to 5000 parts of chitosan; and
250 to 2000 parts of sodium tripolyphosphate;
wherein, in said pharmaceutical carrier, said negatively charged polymer, said chitosan, and said tripolyphosphate combine with each other via electrostatic attraction.

2. The pharmaceutical carrier of claim 1, wherein said initial mixture comprises:
100 parts of a negatively charged polymer;
2400 to 2600 parts of chitosan; and
900 to 1100 parts of sodium tripolyphosphate.

3. The pharmaceutical carrier of claim 1, wherein said pharmaceutical carrier has a particle size of 100 to 200 nm.

4. The pharmaceutical carrier of claim 1, wherein said pharmaceutical carrier is used for carrying a drug for inhibiting Helicobacter pylori.

5. The pharmaceutical carrier of claim 1, wherein said negatively charged polymer is alginate, heparin, polyacrylic acid, or a combination thereof.

6. A drug structure, prepared by an initial mixture, wherein said initial mixture comprises:
100 parts of a negatively charged polymer;
625 to 5000 parts of chitosan;
250 to 2000 parts of sodium tripolyphosphate; and
500 to 4000 parts of an active ingredient
wherein, in said pharmaceutical carrier, said negatively charged polymer, said chitosan, said tripolyphosphate, and said active ingredient combine with each other via electrostatic attraction.

7. The drug structure of claim 6, wherein said initial mixture comprises:
100 parts of a negatively charged polymer;
2400 to 2600 parts of chitosan;
900 to 1100 parts of sodium tripolyphosphate; and
1800 to 2200 parts of an active ingredient.

8. The drug structure of claim 6, having an encapsulation efficiency of 45 to 55%.

9. The drug structure of claim 6, having a particle size of 100 to 200 nm.

10. The drug structure of claim 6, wherein said negatively charged polymer is alginate, heparin, polyacrylic acid, or a combination thereof.

11. The drug structure of claim 6, wherein said drug structure does not comprises a proton pump inhibitor or a bismuth.

12. The drug structure of claim 6, wherein said active ingredient has an activity of inhibiting Helicobacter pylori.

13. The drug structure of claim 12, wherein said active ingredient is selected from amoxicillin, clarithromycin, or omeprazole.

14. The drug structure of claim 6, having a retention time in stomach of no less than 24 hours.

15. The drug structure of claim 6, having a surface charge of 20 to 30 mV.

16. The drug structure of claim 6, being an oral medicine.

17. A method for inhibiting Helicobacter pylori, comprising administrating an effective amount of the drug structure of claim 6 to a population of Helicobacter pylori.

18. The method of claim 17, consisting essentially of the following step: administrating an effective amount of the drug structure of claim 6 to a population of Helicobacter pylori.

19. The method of claim 17, not comprising administrating another substance having an activity of inhibiting Helicobacter pylori to said population of Helicobacter pylori; wherein said substance having an activity of inhibiting Helicobacter pylori is different from said active ingredient of said drug structure.

20. The method of claim 17, not comprising administrating a substance having an auxiliary activity of inhibiting Helicobacter pylori to said population of Helicobacter pylori; wherein said substance having an auxiliary activity of inhibiting Helicobacter pylori is different from the ingredients of said drug structure.

21. The method of claim 20, wherein said substance having an auxiliary activity of inhibiting Helicobacter pylori is proton pump inhibitor or bismuth.

22. The method of claim 17, wherein said effective amount is 1 to 10 mg/kg/day
